(19) United States
(12) Patent Application Publication
(10) Pub. No.: US 2001/0009369 A1
(43) Pub. Date: Jul. 26, 2001

(54) MR data acquisition method, MR image display method and MRI apparatus

(76) Inventors: Yukitoshi Shimo, Tokyo (JP); Yasushi Kato, Tokyo (JP); Takashi Ishiguro, Tokyo (JP)

Correspondence Address: MOONRAY KOJIMA, BOX 627, WILLIAMSTOWN, MA, 01267, US

(21) Appl. No.: 09/757,331

(22) Filed: Jan. 9, 2001

(30) Foreign Application Priority Data
Jan. 20, 2000 (JP) 2000-11394

(51) Int. Cl. G01V 3/00; A61B 5/05

(52) U.S. Cl. 324/307; 324/309; 324/318

(57) ABSTRACT

In order to acquire MR data with respect to a wider area of the subject to be examined at a higher SNR by positioning RF coil for once, an MRI apparatus is provided, which comprises a stage for mounting a subject to be examined, a magnetostatic coil for generating a magnetostatic field, partial each having narrower sensitivity area than a uniform static field and juxtaposed along with the direction of body axis of the subject, a stage movement controller unit for moving the stage so as to include the sensitivity area of one of partial coils in the uniform magnetostatic field to receive NMR signals and for thereafter repeating moving the stage again so as to include another one of partial coils into the uniform magnetostatic field, a coil switching controller unit selectively connecting one among partial coils of which the sensitivity area is included in the uniform magnetostatic field, a computer for generating an MR image based on the MR data, and for synthesize each of MR image by corresponding to the location of the imaging site, and a display device for displaying MR images.

### BACKGROUND OF THE INVENTION

The present invention is related to a MR (magnetic resonance) data acquisition method, an MR image display method, and an MRI apparatus, more specifically to an MR data acquisition method, which allows MR data with respect to a wide region in a subject to be examined to be acquired at a higher SNR (signal to noise ratio) by setting RF coils at once, an MR image display method for displaying an MR image by using the MR data acquired in accordance with the above MR data acquisition method, as well as an MRI (magnetic resonance imaging) apparatus.

FIG. 1 is a schematic diagram of imaging of the cervix by means of an MRI apparatus 500 of the Related Art.

A subject H to be examined will be mounted on a stage 101, will have a partial coil 21A fit to the cervix, and then will be inserted into the bore A of a magnet assembly.

Then a magnetostatic coil 102 will generate a static field Bo, the gradient coil (not shown in the figure) will apply a gradient field, the partial coil 21A will apply RF (radio frequency) pulses, while receiving NMR (nuclear magnetic resonance) signals for gathering MR data. When the sensitivity area αa of the partial coil 21A is narrower than the uniform magnetostatic field area R formed by the magnetostatic coil 102, MR data may be acquired at higher SNR.

FIG. 2 is an explanation when abdominal imaging is to be performed after cervical imaging with the above-mentioned MRI apparatus 500.

The partial coil 21A will be detached from the cervix of the subject H and a partial coil 21B will be put on the abdomen of the subject H. In this case also the sensitivity area αb of the partial coil 21B is narrower than the uniform magnetostatic field R so that the MR data may be gathered at higher SNR.

FIG. 3 is an explanation when the entire spine is to be imaged by means of the MRI apparatus 500.

The subject H will be mounted on the stage 101, then put on with a partial coil 21C on the abdomen, and inserted into the bore A of the magnet assembly.

Then a magnetostatic coil 102 will generate a static field Bo, the gradient coil (not shown in the figure) will apply a gradient field, the partial coil 21C will apply RF (radio frequency) pulses while receiving NMR (nuclear magnetic resonance) signals for gathering MR data. Imageable FOV (field of view) will be enlarged because the sensitivity area αc of the partial coil 21C is wider than the uniform magnetostatic field R, however the SNR of MR data will be aggravated.

In the MRI apparatus 500 above of the Related Art, when used for imaging a wider site of the subject H, the MR data at higher SNR may be gathered if the partial coils 21A and 21B (see FIG. 1 and FIG. 2) having narrower sensitivity area αa or α b, respectively, however there may remain a problem on the time-consuming labor of attaching and detaching a plurality of partial coils. On the other hand, the partial coil may be attached only once if such a partial coil 21C having wider sensitivity area αc (see FIG. 3) is used, leading to a problem of degrading the SNR of MR data.

### SUMMARY OF THE INVENTION

The object of the present invention is to provide an MR data acquisition method which allows MR data with respect to a wider site in a subject being examined to be gathered once the RF coil is attached, an MR image display method and an MRI apparatus for displaying MR images using the MR data gathered by the MR data acquisition method.

[Means for Solving the Problem]

In a first aspect in accordance with the present invention, an MR data acquisition method is provided, which is characterized by placing a plurality of RF coils having a narrower sensitivity area than an uniform magnetostatic field or RF coil groups made of a combination of two or more RF coils along with a subject to be examined, receiving NMR signals from the subject to be examined while positioning one of the RF coils or the RF coil groups within the uniform magnetostatic field for acquiring MR data, and repeating receiving thereafter NMR signals from the subject to be examined while positioning another RF coil or RF coil group within the uniform magnetostatic filed for acquiring MR data until any necessary MR data will have been acquired.

In accordance with the MR data acquisition method of the first aspect above, the reception of NMR signals will be iteratively repeated by sequentially repositioning each of the RF coils or the RF coil groups one at a time, so that the MR data with respect to a wider site of the subject may be acquired once a plurality of RF coils or RF coil groups may have been installed around the subject before imaging. Also, since NMR signals may be received with the sensitivity area of the RF coils or RF coil groups being included within the uniform magnetostatic field, MR data may be gathered at higher SNR.

In a second aspect in accordance with the present invention, an MR data acquisition method is provided, which is characterized by, in the MR data acquisition method set forth in the above first aspect, positioning the RF coils or the RF coil groups so as to form a continuous sensitivity area.

In accordance with the MR data acquisition method of the second aspect above, since the RF coils or RF coil groups are positioned so as to cover the site to be imaged continuously with the sensitivity area, MR data with respect to the site of the subject to be examined may be gathered from a wider region without any gap.

In a third aspect in accordance with the present invention, an MR data acquisition method is provided, which is characterized by, in the MR data acquisition method set forth in the first or second aspect above, performing automatic switching for enabling the RF coils or the RF coil groups positioned in the uniform magnetostatic field in cooperation with the positioning control of the RF coils or the RF coil groups.

In accordance with the MR data acquisition method of the third aspect above, the automatic switching for enabling the RF coils or RF coil groups without intervention of operator allows the work burden of operator to be reduced.

In a fourth aspect in accordance with the present invention, an MR image display method is provided, which is characterized by placing a plurality of RF coils having a narrower sensitivity area than an uniform magnetostatic field or RF coil groups made of a combination of two or more RF coils along with a subject to be examined, receiving NMR signals from the subject to be examined while positioning one of the RF coils or the RF coil groups within the uniform magnetostatic field for acquiring MR data, and repeating receiving thereafter NMR signals from the subject to be examined while positioning another RF coil or RF coil group within the uniform magnetostatic filed for acquiring MR data, and displaying an MR image generated based on the MR data acquired.

In accordance with the MR image display method of the fourth aspect above, an MR image with respect to a broader site of the subject may be displayed at a higher resolution, based on the MR data gathered in accordance with the MR data acquisition method of the first aspect above.

In a fifth aspect in accordance with the present invention, an MR image display method is provided, which is characterized by, in the MR image display method set forth in the fourth aspect, positioning the RF coils or the RF coil groups so as to form a continuous sensitivity area.

In accordance with the MR image display method of the fifth aspect above, an MR image in correspondence with a broader site of the subject may be displayed at a higher resolution, since MR data with respect to the subject to be examined may be gathered from wider range without any gap.

In a sixth aspect in accordance with the present invention, an MR image display method is provided, which is characterized by, in the MR image display method set forth in the fifth aspect, performing switching for enabling the RF coils or the RF coil groups positioned in the uniform magnetostatic field, in correspondence with operation by an operator.

In accordance with the MR image display method of the sixth aspect above, since the switching for enabling the RF coils or the RF coil groups may be performed manually by the operation of an operator, the switching mechanism may be simplified to reduce cost.

In a seventh aspect in accordance with the present invention, an MR image display method is provided, which is characterized by, in the MR image display method set forth in the fourth aspect above or the fifth aspect above, performing automatic switching for enabling the RF coils or the RF coil groups positioned in the uniform magnetostatic field in cooperation with the positioning control of the RF coils or the RF coil groups.

In accordance with the MR image display method of the seventh aspect above, the automatic switching for enabling the RF coils or RF coil groups without intervention of operator allows the work burden of operator to be reduced.

In an eighth aspect in accordance with the present invention, an MR image display method is provided, which is characterized by, in the MR image display method set forth in any one of the fourth aspect above through the seventh aspect above, performing switching for enabling the RF coils or the RF coil groups positioned in the uniform magnetostatic field, in correspondence with operation by an operator.

In accordance with the MR image display method of the eighth aspect above, since the switching for enabling the RF coils or the RF coil groups may be performed manually by the operation of an operator, the switching mechanism may be simplified to reduce cost.

In a ninth aspect in accordance with the present invention, an MR image display method is provided, which is characterized by, in the MR image display method set forth any one of the fourth aspect above through the seventh aspect above, performing automatic positioning control for positioning the RF coils or the RF coil groups in the uniform magnetostatic field in cooperation with the completion of immediately preceding MR data acquisition.

In accordance with the MR image display method of the ninth aspect above, the automatic positioning control of the RF coils or the RF coil groups without intervention of operator allows the work burden of operator to be reduced.

In a tenth aspect in accordance with the present invention, an MR image display method is provided, which is characterized by, in the MR image display method set forth in any one of the fourth aspect above through the ninth aspect above, generating an MR image from each agglomeration of MR data acquired separated in a plurality of times, synthesizing these MR images altogether while corresponding each of images to the respective location of imaging site to create a synthesized MR image, and displaying the synthesized MR image.

In accordance with the MR image display method of the tenth aspect above., many imaging sites over the subject may be displayed in one single image, allowing the entirety of condition of the imaged sites to be read at once.

In an eleventh aspect in accordance with the present invention, an MR image display method is provided, which is characterized by, in the MR image display method set forth in any one of the fourth aspect above through the tenth aspect above, accepting from the operator whether displaying in a tiled fashion on a screen each of MR images based on the MR data gathered in numbers, or displaying the synthesized image, or displaying each of the MR images by switching therebetween.

In accordance with the MR image display method of the eleventh aspect above, MR images may be displayed in an image display manner desired by the operator. More specifically, when displaying each MR image in a tiled fashion, there will be an advantage that the change among MR images derived from different imaged sites may be readily recognized. When displaying a synthesized MR image, there will be an advantage that the entirety of condition of the imaged site may be recognized at once. When displaying each MR image by switching therebetween there will be an advantage that details may be readily read since an MR image may be displayed larger on the screen.

In a twelfth aspect in accordance with the present invention, an MRI apparatus is provided, which is characterized by comprising: a stage having the functionality of moving a subject to be examined mounted thereon, magnetostatic field generator means for generating a static magnetic field, gradient magnetic field applicator means for applying a gradient field, an array of a plurality of RF coils or of RF coil groups each having narrower sensitivity area than a uniform static field formed by the magnetostatic field generator means as well as juxtaposed in the direction of body axis of the subject to be examined, an MR data acquisition controller means for moving the stage such that the sensitivity area of one of the RF coils or of the RF coil groups may be included within the uniform magnetostatic field, receiving NMR signals from the subject to be examined, and for repeating thereafter moving again the stage such that the sensitivity area of another RF coil or RF coil group may be included within the uniform magnetostatic field and receiving NMR signals from the subject to be examined in order to acquire MR data, MR image generator means for generating an MR image based on the acquired MR data, and MR image display means for displaying the MR image.

In accordance with the MRI apparatus of the twelfth aspect above, since the MR image display method set forth in the fourth aspect above is implemented in order to display an MR image, an MR image with respect to a broader site of the subject may be displayed at higher resolution.

In a thirteenth aspect in accordance with the present invention, an MRI apparatus is provided, which is characterized by, in the MRI apparatus set forth in the twelfth aspect above, the RF coils or the RF coil groups being positioned so as to form a continuous sensitivity area.

In accordance with the MRI apparatus of the thirteenth aspect above, an MR image that correspond to a broader and continuous site may be displayed at a higher resolution.

In a fourteenth aspect in accordance with the present invention, an MRI apparatus is provided, which is characterized by, in the MRI apparatus set forth in the twelfth aspect above or the thirteenth aspect above, the MR data acquisition controller means performing control by moving the stage so as to position within the uniform magnetostatic field the region to which contrast medium injected into the subject may reach and receiving NMR signals from the RF coils or the RF coil groups the sensitivity area of which is included in the uniform magnetostatic field.

In accordance with the MRI apparatus of the fourteenth aspect, the stage will be moved in correspondence with the region to which the contrast medium will have reached, resulting in an advantage in reading of distribution of body fluid transporting vascular (in general, blood vessel) within the subject.

In a fifteenth aspect in accordance with the present invention, an MRI apparatus is provided, which is characterized by comprising, in the MRI apparatus set forth in the twelfth aspect above through the fourteenth aspect above, MR image generator means for generating an MR image for each agglomeration of MR data acquired separately in a plurality of times, synthetic MR image generator means for creating a synthetic MR image by synthesizing thus generated MR images by corresponding each to their respective location of imaging site, and a synthetic MR image display means for displaying the synthetic MR image.

In accordance with the MRI apparatus of the fifteenth aspect above, a wide region of imaging site within the subject to be examined may be displayed as one single image, allowing the entirety of condition of the imaged site to be read at once.

In an sixteenth aspect in accordance with the present invention, an MRI apparatus is provided, which is characterized by comprising, in the MRI apparatus set forth in any one of the twelfth aspect above through the fifteenth aspect above, a display mode specifying means for accepting from the operator whether displaying in a tiled fashion on a screen each of MR images based on the MR data gathered in numbers, or displaying the synthesized image, or displaying each of the MR images by switching therebetween.

In accordance with the MRI apparatus of the sixteenth aspect above, MR images may be displayed in an image display mode desired by the operator. More specifically, when displaying each MR image in a tiled fashion, there will be an advantage that the change among MR images derived from different imaged sites may be readily recognized. When displaying a synthesized MR image, there will be an advantage that the entirety of condition of the imaged site may be recognized at once. When displaying each MR image by switching therebetween there will be an advantage that details may be readily read since an MR image may be displayed larger on the screen.

Therefore, in accordance with the MR data acquisition method of the present invention, without time-consuming labor of repositioning RF coils, FOV that allows MR data to be gathered at a higher SNR may be set wider than the uniform magnetostatic field, allowing MR data with respect to a wider area of site(s) (which maybe a single site or a plurality of sites) to be acquired in a shorter period of time at a higher precision.

In addition, in accordance with the MR image display method and the MRI apparatus of the present invention, The MR data above may be used for display an MR image of higher resolution. In particular, when displaying a synthetic MR image that combines each MR image to the corresponding location of the imaged site, instant recognition of entire distribution of spines and vessels may lead to a clinically higher availability.

Further objects and advantages of the present invention will be apparent from the following description of the preferred embodiments of the invention as illustrated in the accompanying drawings.

### BRIEF DESCRIPTION OF THE DRAWINGS

FIG. 1 is an explanation diagram of imaging of the cervix by means of an MRI apparatus of the Related Art.

FIG. 2 is an explanation when abdominal imaging is performed followed by cervical imaging with the MRI apparatus of the Related Art.

FIG. 3 is an explanation when the entire spine is imaged by means of an MRI apparatus of the Related Art.

FIG. 4 is a schematic block diagram indicating an MRI apparatus in accordance with one preferred embodiment of the present invention.

FIG. 5 is a schematic block diagram indicating an MR image display process by the MRI apparatus shown in FIG. 1.

FIG. 6 is an explanation diagram indicating the state that the stage is moved such that the sensitivity area of first partial coil is included in the uniform magnetostatic field.

FIG. 7 is an explanation diagram indicating the state that the stage is moved such that the sensitivity area of second partial coil is included in the uniform magnetostatic field.

FIG. 8 is a schematic diagram indicating the state that the stage is moved such that the sensitivity area of fifth partial coil is included in the uniform magnetostatic field.

FIG. 9 is a schematic diagram indicating a display that each MR images is tiled in one screen.

FIG. 10 is a schematic diagram indicating the display of a synthetic MR image.

FIG. 11 is a schematic diagram indicating the switchable mode display of each MR image.

### DETAILED DESCRIPTION OF THE INVENTION

The present invention will be described in greater details herein below with reference to a preferred embodiment shown in the drawings. It should be noted that it is presented merely for the purpose of illustration and is not intended to be exhaustive or to limit the invention to the precise form disclosed.

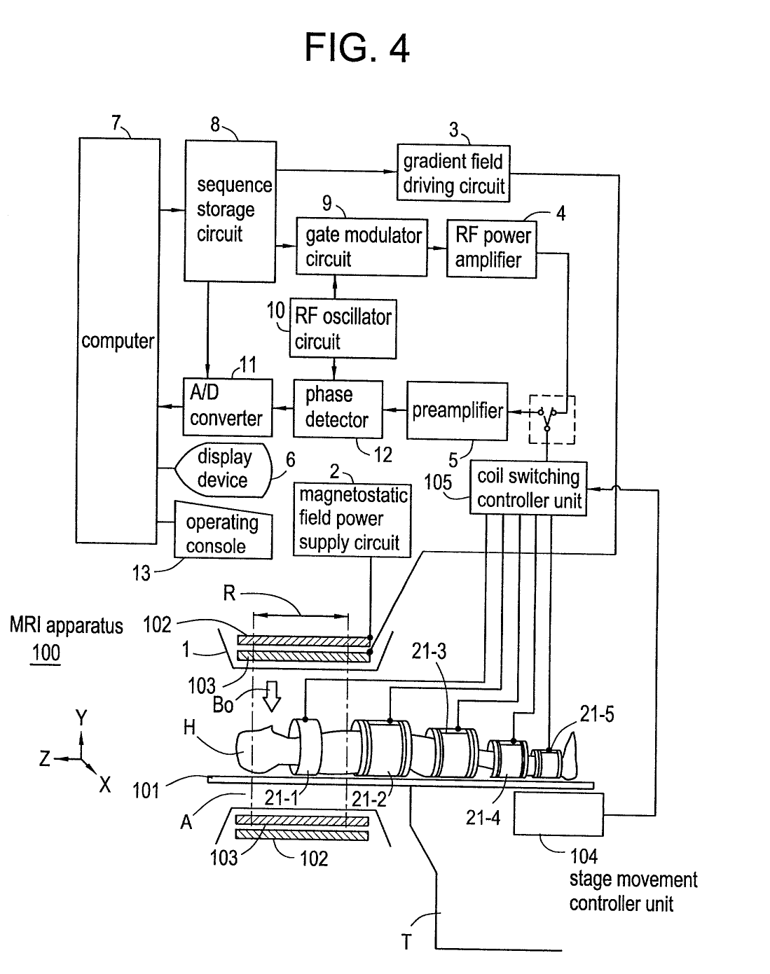

FIG. 4 shows a schematic block diagram of an MRI apparatus 100 in accordance with a preferred embodiment of the present invention.

In the MRI apparatus 100, a magnet assembly 1 has a bore A for inserting therein a subject H to be examined mounted on a stage 101 on an imaging table T, and has a magnetostatic coil 102 for generating a predefined magnetostatic field Bo (field length of 0.5 T-1.5 T, for example) and a gradient field coil 103 for generating a gradient field respectively in x-axis, y-axis, and z-axis, both coils surrounding the bore A. The movement of the stage 101 may be controlled by a stage movement controller unit 104.

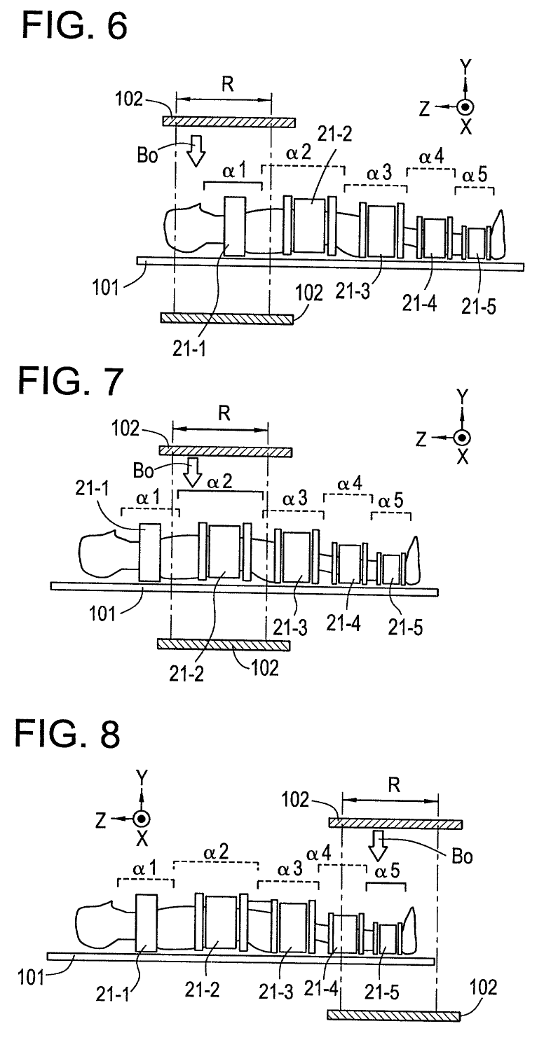

On the cervix, abdomen, coxa, genu, and foot of the subject H, partial coils for transmitting RF pulses and receiving NMR signals 21-1, 21-2, 21-3, 21-4, and 21-5 respectively are placed. the partial coils 21-1 through 21-5 have each a sensitive area (α1 through α5 in FIG. 6 to FIG. 8) that is narrower than the uniform magnetostatic field R formed by the magnetostatic coil 102. the partial coils 21-1 through 21-5 maybe of, for example, solenoid coils, or combination coils of solenoid coils and saddle coils.

The magnetostatic coil 102 is connected to a magnetostatic field power supply circuit 2, while the gradient field coil 103 is connected to a gradient field driving circuit 3.

A coil switching controller unit 105, in cooperation with the operation of the stage movement controller unit 104, may control the selective connection of coil having its sensitivity area in the uniform magnetostatic field R among partial coils 21-1 through 21-5 to an RF power amplifier 4 and a preamplifier 5.

A sequence storage circuit 8, in response to instructions from a computer 7, may operate the gradient field driving circuit 3 based on the stored pulse sequences to cause the gradient field coil 103 to generate a gradient field, as well as operate a gate modulator circuit 9 to modulate carrier output signals from an RF oscillator circuit 10 into pulse signal in a form of predetermined envelope at a predetermined timing, then amplify the power thereof with the RF power amplifier 4 to supply it to any of partial coils 21-1 through 21-5 selected by the coil switching controller unit 105, in order to selectively excite the region to be imaged of the subject H.

The preamplifier 5 may amplify NMR signals received from the subject H by any of the partial coils 21-1 through 21-5 to input to a phase detector 12. The phase detector 12 uses the carrier output signals from the RF oscillator circuit 10 as the reference signal to phase demodulate the NMR signals from the preamplifier 5 to feed to an A/D converter 11. The A/D converter 11 may convert analog signals derived from the phase demodulation into digital signals to input to the computer 7.

The computer 7 may store the input digital signals as MR data, and reconstruct an image by performing image reconstruction process when MR data sufficient to construct a single MR image. The computer 7 may also correspond and combine the MR image based on the MR data gathered separately in a plurality of times to the location of imaged site. These MR images will be displayed on a display device 6. Furthermore, the computer 7 is served for the overall control including such as receiving information input from an operating console 13.

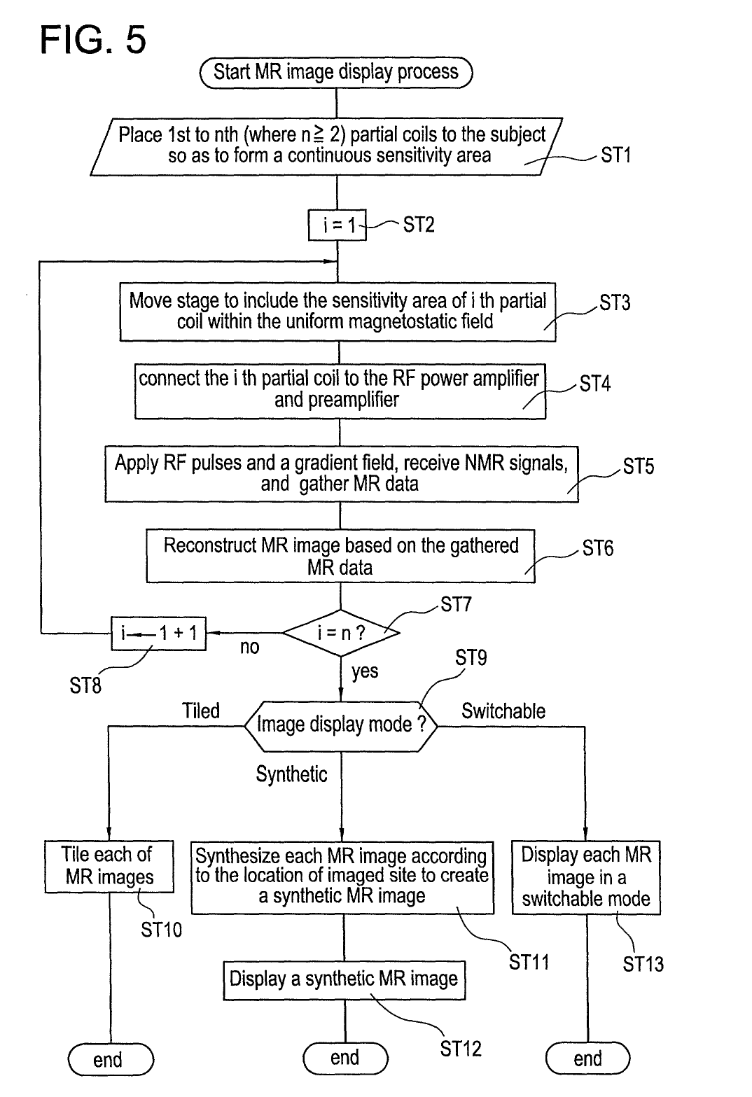

FIG. 5 is a flow diagram indicating MR image display process performed by this MRI apparatus 100. Total processing time required for this MR image display process may be for example approximately 1 minute to 20 minutes.

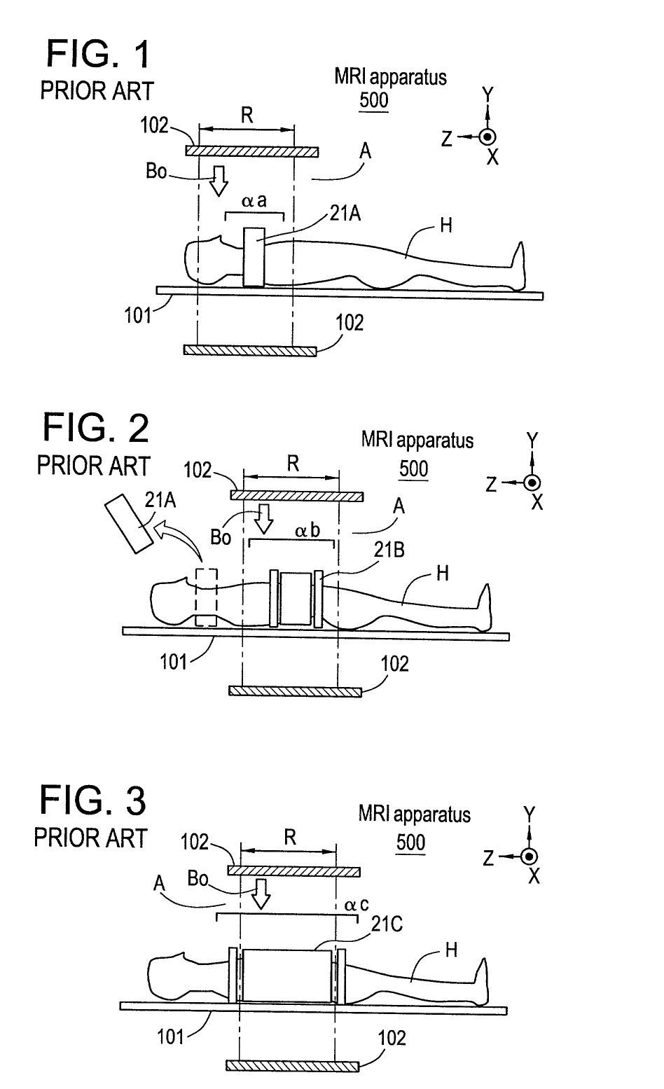

In step ST1, 1st to nth (where n≧2) partial coils will be placed, along with the body axis of the subject H such that the sensitivity area will continuously cover necessary site to be imaged. In general, a partial coil has a sensitivity area somewhat wider than its size. In the example shown in FIG. 1, partial coils 21-1 through 21-5 (in this case, n=5) will be placed.

In step ST2, partial coil i is initialized to ‘1’. For the purpose of illustration, the number i of partial coil of the partial coils 21-1 through 21-5 are to be designated to 1 to 5.

In step ST3, as shown in FIG. 6, the stage 101 will be moved such that the sensitivity area of ith partial coil (if i=1, then the sensitivity area α1 of the partial coil 21-1) will be included within the uniform magnetostatic field R.

In step ST4, the coil switching controller unit 105 will perform control so as to connect the ith partial coil to the RF power amplifier 4 and the preamplifier 5.

Instep ST5, RF pulses and a gradient field will be applied to the subject H, and then the NMR signals from the ith partial coil will be received to acquire MR data. The period of time required for gathering MR data for one pass may be for example approximately 10 seconds.

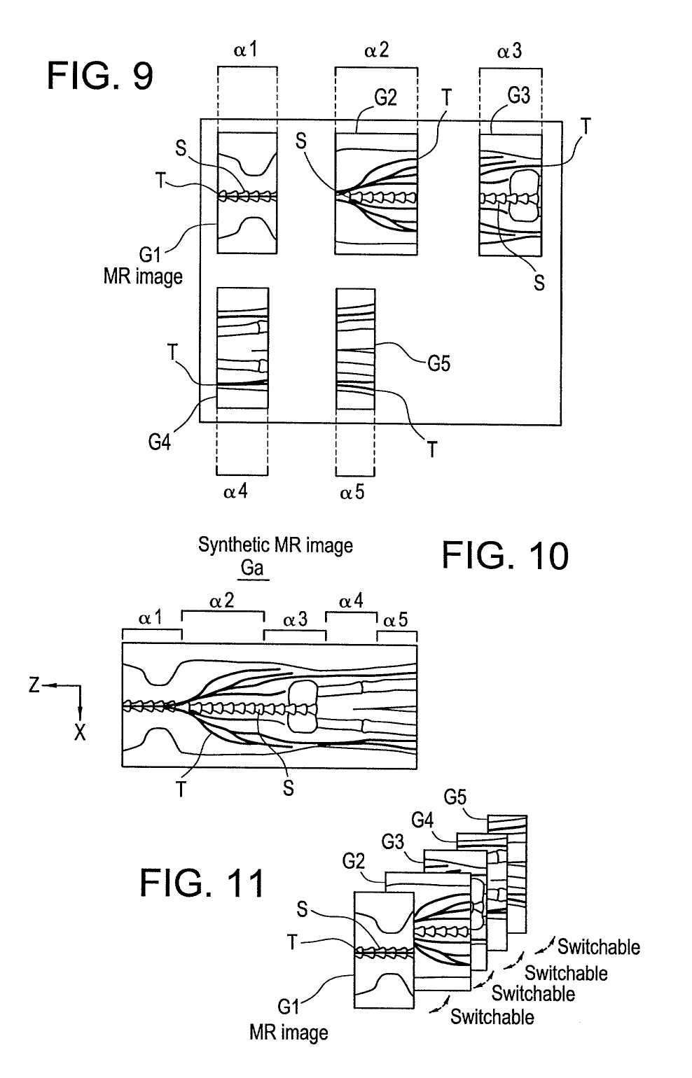

In step ST6, MR images (G1 through G5 shown in FIG. 9 and FIG. 11) will be reconstructed from MR data acquired in the step ST5 above.

Instep ST7, the number i of partial coil will be determined whether or not to be ‘n’. If i=n then the process proceeds to step ST9, if not i=n then proceeds to step ST8.

In step ST8, the number i of partial coil will be incremented by 1, and then the process proceeds to the step ST3 above. The partial coils 21-1 through 21-5 will thereby be sequentially relocated to the uniform magnetostatic field R to repeat the reception of NMR signals. FIG. 7 shows the positional relationship between the uniform magnetostatic field R and the partial coils 21-1 through 21-5 in case of the partial coil number i=2. FIG. 8 shows the positional relationship between the uniform magnetostatic field R and the partial coils 21-1 through 21-5 in case of the partial coil number i=5.

In step ST9, the instructions of image display mode will be received from the operator. If tiling is instructed then the process proceeds to step ST10, if synthetic image display is instructed then proceeds to step ST11, and if MR image switching display is instructed then proceeds to step ST13.

In step S10, as shown in FIG. 9, MR images G1-G5 (each corresponds to sensitivity area α1-α5, respectively) that have been reconstructed in the step ST6 above will be tiled in a screen of display. In the figure, S designates to a spine, T to a blood vessel. This display mode has an advantage that the interpreter of radiogram may readily recognize any alteration in MR images G1 to G5 caused by the difference in imaging site. Thereafter, MR image display process will be terminated.

In step S11, the MR images G1-G5 will be combined by corresponding to the position of imaging site to create a synthetic MR image Ga.

In step S12, as shown in FIG. 10, the synthetic MR image Ga will be displayed. This display mode has an advantage that the distribution of spine S and vessel T in a wider region (for example, approximately 40 centimeters in the direction of body axis) may be recognized instantly. Thereafter, MR image display process will be terminated.

In step S13, as shown in FIG. 11, each of MR images G1-G5 will be displayed in a switchable mode. This display mode, which may display the MR images G1-G5 larger on the display, has an advantage that details may be readily read out. Thereafter, MR image display process will be terminated.

In accordance with the MRI apparatus 100 as have been described above, since NMR signals may be iteratively received by sequentially repositioning the partial coils 21-1 through 21-5 into the uniform magnetostatic field R, MR data with respect to a wider region of the subject H may be gathered at a higher SNR if the partial coils 21-1 through 21-5 are placed only once prior to imaging, so that MR image at a higher resolution will be displayed.

It is to be noted here that the configuration of the MRI apparatus 100 above may be modified as follows:

(1) by using the partial coils 21-1 through 21-5 as coils exclusive for reception, other transmitter coils for the RF pulses may be installed in addition thereto.

(2) instead of using the partial coils 21-1 through 21-5, phased-array coils, or a multi-coil that couples a plurality of coils may be used.

(3) the operator may manually move the stage 101, or switch between the partial coils 21-1 through 21-5. In this case, movement or switching mechanism may be simplified to reduce cost.

(4) if contrast medium is injected into the subject H to picturize the image of blood vessel T, NMR signals may be received while moving the stage 101 so as to align the region to which the contrast medium reaches within the uniform magnetostatic field R.

Many widely different embodiments of the invention may be configured without departing from the spirit and the scope of the present invention. It should be understood that the present invention is not limited to the specific embodiments described in the specification, except as defined in the appended claims.

1. An MR data acquisition method comprising the steps of:
placing a plurality of RF coils having a narrower sensitivity area than an uniform magnetostatic field or RF coil groups made of a combination of two or more RF coils along with a subject to be examined;
receiving NMR signals from said subject to be examined while positioning one of said RF coils or said RF coil groups within said uniform magnetostatic field for acquiring MR data; and
repeating receiving thereafter NMR signals from said subject to be examined while positioning another RF coil or RF coil group within said uniform magnetostatic filed for acquiring MR data until any necessary MR data will have been acquired.

2. The MR data acquisition method of claim 1 further comprising the step of: positioning the RF coils or the RF coil groups so as to form a continuous sensitivity area.

3. The MR data acquisition method of claim 1 further comprising the step of: performing automatic switching for enabling the RF coils or the RF coil groups positioned in the uniform magnetostatic field in cooperation with the positioning control of the RF coils or the RF coil groups.

4. An MR image display method comprising the steps of:
placing a plurality of RF coils having a narrower sensitivity area than an uniform magnetostatic field or RF coil groups made of a combination of two or more RF coils along with a subject to be examined;
receiving NMR signals from said subject to be examined while positioning one of said RF coils or said RF coil groups within said uniform magnetostatic field for acquiring MR data;
repeating receiving thereafter NMR signals from said subject to be examined while positioning another RF coil or RF coil group within said uniform magnetostatic filed for acquiring MR data; and
displaying an MR image generated based on the MR data acquired.

5. The MR image display method of claim 4 further comprising the step of: positioning the RF coils or the RF coil groups so as to form a continuous sensitivity area.

6. The MR image display method of claim 4 further comprising the step of: performing switching for enabling the RF coils or the RF coil groups positioned in the uniform magnetostatic field, in correspondence with operation by an operator.

7. The MR image display method of claim 4 further comprising the step of: performing automatic switching for enabling the RF coils or the RF coil groups positioned in the uniform magnetostatic field in cooperation with the positioning control of the RF coils or the RF coil groups.

8. The MR image display method of claim 4 further comprising the step of: performing switching for enabling the RF coils or the RF coil groups positioned in the uniform magnetostatic field, in correspondence with operation by an operator.

9. The MR image display method of claim 4 further comprising the step of: performing automatic positioning control for positioning the RF coils or the RF coil groups in the uniform magnetostatic field in cooperation with the completion of immediately preceding MR data acquisition.

10. The MR image display method of claim 4 further comprising the steps of:
generating an MR image from each agglomeration of MR data acquired separately in a plurality of times;
synthesizing these MR images altogether while corresponding each of them to their respective location of imaging site to create a synthesized MR image; and
displaying the synthesized MR image.

11. The MR image display method of claim 4 further comprising the step of: accepting from the operator whether displaying in a tiled fashion on a screen each of MR images based on the MR data gathered in numbers, or displaying the synthesized image, or displaying each of the MR images by switching therebetween.

12. An MRI apparatus comprising:
a stage having the functionality of moving a subject to be examined mounted thereon;
magnetostatic field generator means for generating a static magnetic field;
gradient magnetic field applicator means for applying a gradient field;
an array of a plurality of RF coils or of RF coil groups each having narrower sensitivity area than a uniform static field formed by said magnetostatic field generator means as well as juxtaposed in the direction of body axis of said subject to be examined;
an MR data acquisition controller means for moving said stage such that the sensitivity area of one of said RF coils or of said RF coil groups may be included within said uniform magnetostatic field, receiving NMR signals from said subject to be examined, and for repeating thereafter moving again said stage such that the sensitivity area of another RF coil or RF coil group may be included within said uniform magnetostatic field and receiving NMR signals from said subject to be examined in order to acquire MR data;
MR image generator means for generating an MR image based on the acquired MR data; and
MR image display means for displaying said MR image.

13. The MRI apparatus of claim 12, wherein the RF coils or the RF coil groups are positioned so as to form a continuous sensitivity area.

14. The MRI apparatus of claim 12 further comprising: the MR data acquisition controller means performing control by moving the stage so as to position within the uniform magnetostatic field the region to which contrast medium injected into the subject may reach and receiving NMR signals from the RF coils or the RF coil groups the sensitivity area of which is included in the uniform magnetostatic field.

15. The MRI apparatus of claim 12 further comprising:
MR image generator means for generating an MR image from each agglomeration of MR data that have been acquired for a plurality of times;
synthetic MR image generator means for creating a synthetic MR image by synthesizing thus generated MR images by corresponding each to their respective location of imaging site; and
synthetic MR image display means for displaying said synthetic MR image.

16. The MRI apparatus of claim 12 further comprising: display mode specifying means for accepting from the operator whether displaying in a tiled fashion on a screen each of MR images based on the MR data gathered in numbers, or displaying the synthesized image, or displaying each of the MR images by switching therebetween.
